(19) United States
(12) Patent Application Publication
(10) Pub. No.: US 2014/0057321 A1
(43) Pub. Date: Feb. 27, 2014

(54) METHOD FOR REDUCING GREENHOUSE GASES

(71) Applicants: HYUNDAI MOTOR COMPANY, Seoul (KR); KIA MOTORS CORPORATION, Seoul (KR)

(72) Inventors: Shin Tae Bae, Anyang (KR); Jeong Kyu Park, Seongnam (KR); Won Bae Lee, Seoul (KR); Dae Young Goh, Busan (KR)

(73) Assignees: Kia Motors Corporation, Seoul (KR); Hyundai Motor Company, Seoul (KR)

(21) Appl. No.: 13/690,558

(22) Filed: Nov. 30, 2012

(30) Foreign Application Priority Data
Aug. 21, 2012 (KR) 10-2012-0091305

(51) Int. Cl. C12P 7/64

(52) U.S. Cl. CPC C12P 7/649
USPC 435/71.1; 435/134; 435/105; 435/159

(57) ABSTRACT

The present invention provides a method of reducing greenhouse gases by capturing and storing carbon dioxide in the form of a biomass, which may be converted into a high value material such as, for example, an oil having more than 37% of omega-3, biodiesel, phospholipid, glycerin, glucose, and a protein feed.

### CROSS-REFERENCE TO RELATED APPLICATION

This application claims under 35 U.S.C. §119(a) the benefit of Korean Patent Application No. 10-2012-0091305, filed Aug. 21, 2012, the entire contents of which are incorporated herein by reference.

### BACKGROUND

(a) Technical Field

The present disclosure relates to a method for reducing greenhouse gases and, more particularly, to reducing greenhouse gases through carbon dioxide capture, fixation, and conversion.

(b) Background Art

As global environmental problems such as global warming and exhaustion of fossil fuels due to the heavy use of fossil fuels arise, a variety of methods for solving these problems have been suggested. Conventional carbon capture & storage (CCS) methods include capturing carbon dioxide from carbon dioxide sources (e.g., thermal power plants, steel mills, and boilers) by using absorption, adsorption, membrane separation, and the like, and transporting the captured carbon dioxide to underground or marine oil reservoirs, gas reservoirs, or coal beds to inject and store the carbon dioxide therein. Although these methods directly reduce green-house gases, costs for capturing, transporting, and storing 1 ton of carbon dioxide are $60-70, $1-10, and $2-10, respectively. In addition to methods of capturing and storing carbon dioxide, methods for converting carbon dioxide into a biomass, such as, e.g., methane, methanol, plastics (e.g., polycarbonate, carbonates, and the like) have been developed. However, improved values of these products in terms of greenhouse gas reduction are far lower than the costs associate with capturing carbon dioxide. FIG. 1 shows a conventional method of capturing and storing carbon dioxide.

One conventional method for reducing greenhouse gases discloses a system for fixing carbon dioxide using microalgae that includes a gas capturing device for capturing carbon dioxide and a photobio reactor for culturing microalgae by receiving carbon dioxide and water.

The gas capturing may be performed by a wet process, and the system may further include a biomass for storing the microalgae cultured in the photobio reactor. However, the value of the captured and stored carbon dioxide is limited because the system does not include a method and device for converting carbon dioxide into a non-detrimental form. Additionally, the operating requirements of such a conventional system for fixing carbon dioxide prevent the system from being broadly applicable in industrial settings. As the global increase in greenhouse gas levels is predicted to have serious detrimental environmental consequences at the global level, there is an urgent need for methods and apparatus that reduce, eliminate, and/or mitigate greenhouse gas production.

### SUMMARY OF THE DISCLOSURE

The present invention provides a method of capturing and storing carbon dioxide by which greenhouse gases may be reduced by capturing carbon dioxide and high value-added materials may be obtained by converting the captured carbon dioxide into functional oil having more than 37% of omega-3, phospholipid, biodiesel, glucose, and the like.

In a preferred exemplary embodiment, the present invention provides a method of preparing high value-added materials from carbon dioxide by capturing and fixing carbon dioxide to obtain a biomass (e.g., C₆H₁₂O₆), and converting the biomass.

The carbon dioxide may be captured by a process of chemically absorbing carbon dioxide through a gas-liquid phase contact between the carbon dioxide-containing exhaust gas discharged from a carbon dioxide source and a liquid absorbent and isolating carbon dioxide from the liquid absorbent by applying heat to the liquid absorbent.

The carbon dioxide may be fixed by a process of obtaining a biomass (C₆H₁₂O₆) by the growth process of microalgae such as Senedesmus and Chlorella Vulgaris including photosynthesis using the captured carbon dioxide, and then a drying process of the resulting microalgae. The conversion of the biomass (C₆H₁₂O₆) according to the techniques herein may produce functional oil having more than 37 wt % of omega-3, biodiesel, phospholipid, glucose, a protein feed, glycerin, and the like, by using a press to crush the cell walls of the biomass to produce oil or an oil cake.

Other aspects and preferred embodiments of the invention are discussed infra.

### BRIEF DESCRIPTION OF THE DRAWINGS

The above and other features of the present invention will now be described in detail with reference to certain exemplary embodiments thereof illustrated the accompanying drawings which are given hereinbelow by way of illustration only, and thus are not limitative of the present invention, and wherein:

FIG. 1 schematically shows a conventional technique of capturing and storing carbon dioxide;

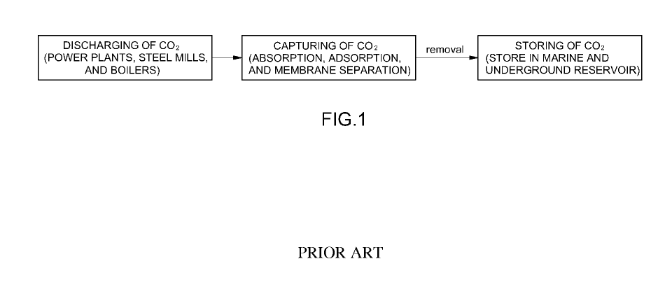

FIG. 2 schematically shows a method of reducing greenhouse gases and creating an added value through capture, fixation, and conversion of carbon dioxide according to an exemplary embodiment of the present invention;

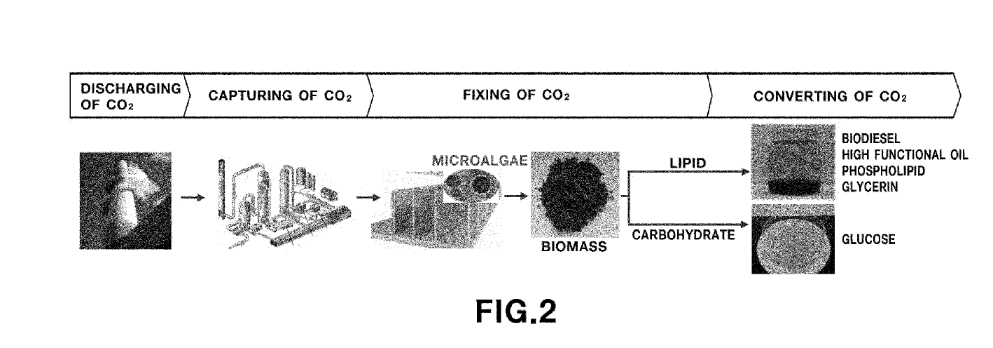

FIG. 3 schematically shows capturing and fixing of carbon dioxide according to an exemplary embodiment of the present invention; and

FIG. 4 is a schematic block diagram illustrating converting of a biomass (C₆H₁₂O₆) according to an exemplary embodiment of the present invention.

It should be understood that the appended drawings are not necessarily to scale, presenting a somewhat simplified representation of various preferred features illustrative of the basic principles of the invention. The specific design features of the present invention as disclosed herein, including, for example, specific dimensions, orientations, locations, and shapes will be determined in part by the particular intended application and use environment.

In the figures, reference numbers refer to the same or equivalent parts of the present invention throughout the several figures of the drawing.

### DETAILED DESCRIPTION

Hereinafter reference will now be made in detail to various embodiments of the present invention, examples of which are illustrated in the accompanying drawings and described below. While the invention will be described in conjunction with exemplary embodiments, it will be understood that the present description is not intended to limit the invention to those exemplary embodiments. On the contrary, the invention is intended to cover not only the exemplary embodiments, but also various alternatives, modifications, equivalents and other embodiments, which may be included within the spirit and scope of the invention as defined by the appended claims.

It is understood that the term “vehicle” or “vehicular” or other similar term as used herein is inclusive of motor vehicles in general such as passenger automobiles including sports utility vehicles (SUV), buses, trucks, various commercial vehicles, watercraft including a variety of boats and ships, aircraft, and the like, and includes hybrid vehicles, electric vehicles, plug-in hybrid electric vehicles, hydrogen-powered vehicles and other alternative fuel vehicles (e.g., fuels derived from resources other than petroleum). As referred to herein, a hybrid vehicle is a vehicle that has two or more sources of power, for example both gasoline-powered and electric-powered vehicles.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

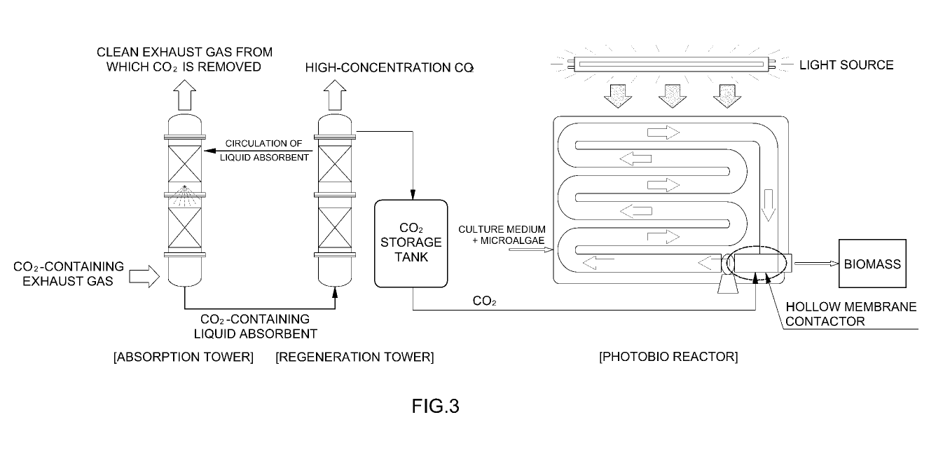

The capture of carbon dioxide may include chemical absorption using a liquid absorbent such as, for example, amine, potassium carbonate, or ammonia water. As shown in FIG. 3, a carbon dioxide-containing exhaust gas discharged from a carbon dioxide source may be chemically absorbed by an absorption tower of a capturing device through a gas-liquid phase contact between the carbon dioxide-containing exhaust gas and a liquid absorbent, preferably, at about 25° C. to about 80° C., so that exhaust gas from which carbon dioxide is removed is discharged. Then, the liquid absorbent chemically bound to the carbon dioxide may be sent to a high-temperature regeneration tower, preferably, at about 60° C. to 150° C., to dissociate the chemical bond and isolate carbon dioxide, so that high-concentration carbon dioxide, preferably, more than about 90% of carbon dioxide may be temporarily stored in a storage tank, or the like, in order to transport carbon dioxide to a carbon dioxide fixing device.

The carbon dioxide may be fixed by photosynthesis by supplying the captured carbon dioxide to a photobio reactor including microalgae and providing the reactor with light energy from a light source and a culture medium, preferably, BG-11, as shown in FIG. 3. More particularly, microalgae such as Senedesmus and Chlorella Vulgaris that actively assimilate carbon may be cultured in an appropriate culture medium and cultured in the photobio reactor, while the captured carbon dioxide is added to the photobio reactor through a hollow membrane contactor used for increasing transfer rates between gas-liquid phase materials, so that the carbon dioxide may be dissolved and saturated in the forms of HCO₃⁻ and CO₃²⁻. The carbon dioxide dissolved in the culture medium may be used as a carbon source to produce a biomass (e.g., C₆H₁₂O₆) by photosynthesis of the microalgae using a light source such as sunlight, a fluorescent lamp, or a light-emitting diode (LED). In this regard, when about 30 to about 40 ppm of the microalgae is cultured in about a 0.05 to about 0.2 M culture medium at about 25□ to about 30° C. using a fluorescent lamp for about 5 to 9 days, the output of the biomass may be in the range of about 200 to about 400 kg per 1 ton of the captured carbon dioxide.

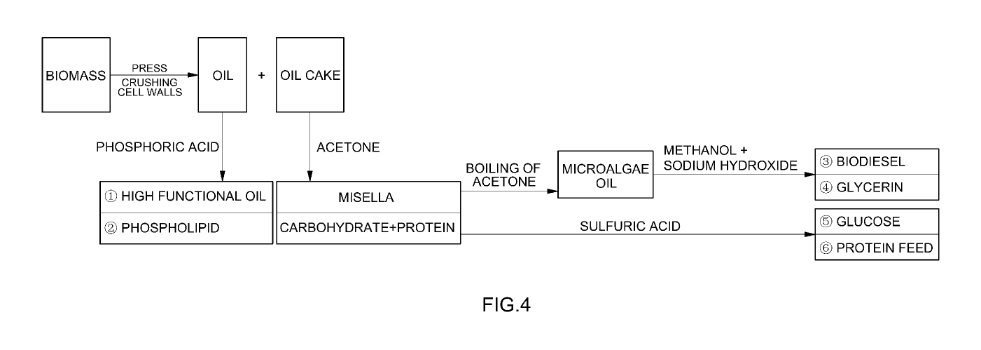

The biomass (e.g., C₆H₁₂O₆) may be converted to obtain oil and oil cake by crushing the cell walls of the biomass using a press as shown in FIG. 4. For example, if about 3 to about 7 ml of a 5% phosphoric acid solution per 1 kg of the biomass is added to the oil, and the mixture is heated for about 10 to 60 minutes at about 70□ to about 100° C. and maintained, a highly functional oil having more than 37 wt % of omega-3 and a phospholipid may be produced. In addition, if about 90 to about 130 ml of an acetone solution per 1 kg of the biomass is added to the oil cake, and the mixture is maintained, misella and a mixture of carbohydrate and protein may be obtained. If the acetone is boiled by heating the misella at about 40□ to 70° C., microalgae oil (extract oil) may be obtained. If about 15 to about 25 ml of methanol and about 0.1 to about 1.0 ml of sodium hydroxide are added to the microalgae oil, and the mixture is heated for about 30 to 60 minutes at about 40□ to about 100° C. and maintained, biodiesel and glycerin may be obtained. Furthermore, if dilute sulfuric acid is added to the mixture of carbohydrate and protein obtained from the oil cake, and the mixture is heated for about 10 to 40 minutes at about 100□ to 150° C., glucose and a protein feed may be produced. In this regard, yields and outputs of the products per 1 kg of the biomass that may be obtained according to the techniques herein are disclosed in Table 1 below.

TABLE 1
Yield and output of materials obtained from
carbon dioxide conversion

| | | |
|---|---|---|
| Materials obtained from | | |
| conversion of carbon dioxide | Yield | Output |

| | | | |
|---|---|---|---|
| Fat (10%) | Functional oil | 90% | 0.090 kg |
| Fat (16%) | Biodiesel | 86% | 0.138 kg |
| | Phospholipid | 8.6% | 0.014 kg |
| | Glycerin | 8.6% | 0.014 kg |
| Carbohydrate (49%) | Glucose | 72% | 0.353 kg |
| Ash (3%) | | 60% | 0.018 kg |
| Protein (22%) | Protein feed | 100% | 0.220 kg |
| Total | | | 0.847 kg |

When 1 ton of carbon dioxide captured according to an embodiment of the present invention is treated, 35 kg of the biomass may be obtained, and outputs and values of value-added materials produced therefrom are disclosed in Table 2 below.

TABLE 2
Output and value of high value-added materials

| High value-added material | Output (kg) | Value ($) |
|---|---|---|
| Functional oil | 31.5 | 146.8 |
| Biodiesel | 48.3 | 48.1 |
| Phospholipid | 4.9 | 9.6 |
| Glycerin | 4.9 | 0.5 |
| Glucose | 123.6 | 129.6 |
| Protein feed | 77 | 35.9 |
| Total | 0.847 | 370.5 |

According to the present invention, high value-added products may be obtained as follows.

Although green-house gases may be reduced according to conventional capturing and storing methods, treatment of 1 ton of carbon dioxide costs $63-90. Although methods for converting carbon dioxide into a biomass, methane, methanol, plastics, e.g., polycarbonate, carbonates, and the like have been developed, improved value of these products is far lower than costs for capturing carbon dioxide.

According to the method of the present invention, greenhouse gases may be reduced, and high value products such as, for example, expensive high functional oil, biodiesel, phospholipid, and glucose ($300 to 420 per 1 ton of carbon dioxide) may be obtained according to the carbon fixation techniques described above. Thus, profits may be greater than expenses ($60 to 70 for the capturing and $170 to 200 for fixing and converting).

The invention has been described in detail with reference to preferred embodiments thereof. However, it will be appreciated by those skilled in the art that changes may be made in these embodiments without departing from the principles and spirit of the invention, the scope of which is defined in the appended claims and their equivalents.

1. A method, comprising:
capturing carbon dioxide from a carbon dioxide source;
fixing the carbon dioxide;
converting the fixed carbon dioxide into a biomass; and
preparing a high value material.

2. The method of claim 1, wherein capturing further comprises:
absorbing the carbon dioxide with a liquid absorbent selected from the group consisting of amine, potassium carbonate, ammonia water, and any combination thereof.

3. The method of claim 1, wherein capturing further comprises:
contacting the carbon dioxide with a liquid absorbent;
chemically absorbing the carbon dioxide in an absorbing tower of a capturing device through a gas-liquid phase contact between the carbon dioxide contained in an exhaust gas discharged from a carbon dioxide-producing source and the liquid absorbent; and
discharging exhaust gas from which the carbon dioxide is removed.

4. The method of claim 3, wherein the carbon dioxide is chemically absorbed at a temperature in the range of 25° C. to 80° C.

5. The method of claim 3, further comprising:
transporting the liquid absorbent and the absorbed carbon dioxide to a high-temperature regeneration tower;
dissociating the carbon dioxide from the liquid absorbent in the high-temperature regeneration tower; and
isolating the dissociated carbon dioxide to a storage tank.

6. The method of claim 5, wherein a temperature of the high-temperature regeneration tower is in the range of 60° C. to 150° C. and the isolated carbon dioxide has a concentration of 90% or greater.

7. The method of claim 1, wherein fixing further comprises:
supplying the captured carbon dioxide to a photobio reactor including microalgae, a light source, and a culture medium; and
producing a biomass,
wherein the light source is sunlight or an artificial light source and the microalgae is Senedesmus or Chlorella Vulgaris.

8. The method of claim 7, wherein the carbon dioxide is supplied to the photobio reactor via a hollow membrane contactor in the form of HCO₃⁻ or CO₃²⁻.

9. The method of claim 7, wherein the biomass is converted to an oil and/or an oil cake by crushing cell walls of the microalgae using a press.

10. The method of claim 9, wherein the oil is treated with a phosphoric acid solution and heated to produce a processed oil having more than 37 wt % of omega-3 or phospholipid.

11. The method of claim 10, wherein 3 to 7 ml of a 3 to 7% phosphoric acid solution per 1 kg of the biomass is added to the oil.

12. The method of claim 10, wherein the oil to which the phosphoric acid solution is added is heated for 10 to 60 minutes at a temperature that ranges from 70° C. to 100° C.

13. The method of claim 9, wherein the oil cake is treated with an acetone solution to produce a misella and/or a mixture of carbohydrate and protein.

14. The method of claim 13, wherein 90 to 130 ml of the acetone solution per 1 kg of the biomass is added to the oil cake.

15. The method of claim 13, wherein the misella is further heated to boil acetone so as to obtain microalgae oil (extract oil).

16. The method of claim 15, wherein the misella is heated to a temperature in the range of 40° C. to 70° C.

17. The method of claim 15, wherein methanol and sodium hydroxide are added to the microalgae oil and the mixture is heated to obtain biodiesel or glycerin.

18. The method of claim 17, wherein 15 to 25 ml of methanol and 0.1 to 1.0 ml of sodium hydroxide are added to the microalgae oil and the mixture is heated for 30 to 60 minutes at 40° C. to 100° C.

19. The method of claim 13, wherein dilute sulfuric acid is added to the mixture of carbohydrate and protein obtained from the oil cake and the mixture is heated to produce glucose or a protein feed, wherein the heating is performed for 10 to 40 minutes at 100 to 150° C.

20. The method of claim 1, wherein the high value material comprises one or more materials selected from the group consisting of a functional oil having more than 37% of omega-3, biodiesel, phospholipid, glycerin, glucose, and a protein feed.
